(19) United States
(12) Patent Application Publication
(10) Pub. No.: US 2026/0272320 A1
(43) Pub. Date: Sep. 17, 2026

(54) IMPLANTABLE PRESSURE SENSOR AND MONITORING SYSTEM

(71) Applicant: AMES LifeSciences Co., Ltd., Chengdu (CN)

(72) Inventors: Binjie Zhu, Chengdu (CN); Xiaojing Zhong, Chengdu (CN); Zehua Lan, Chengdu (CN)

(73) Assignee: AMES LifeSciences Co., Ltd., Chengdu (CN)

(21) Appl. No.: 19/676,335

(22) Filed: May 13, 2026

Related U.S. Application Data
(63) Continuation of application No. PCT/CN2025/128910, filed on Oct. 21, 2025.

(30) Foreign Application Priority Data
Oct. 23, 2024 (CN) 202411485322.X

(51) Int. Cl. A61B 5/03; A61B 5/00; A61B 5/01

(52) U.S. Cl. CPC A61B 5/031; A61B 5/01; A61B 5/6847; A61B 5/6868; A61B 5/742; A61B 2560/0214; A61B 2560/0257; A61B 2562/0247; A61B 2562/0271; A61B 2562/166

(57) ABSTRACT

The present invention provides an implantable pressure sensor and a monitoring sensor. The detection sensor includes an inflatable bag to be implanted into a site to be measured; a first gas pressure sensor disposed in an environment in gas communication with interior of the inflatable bag, for measuring an gas pressure value inside the inflatable bag. Combination of the inflatable bag and the first gas pressure sensor enables implantable pressure monitoring, and reducing volume and size of the detection sensor. Direct measurement of intracranial pressure via the inflatable bag results in accurate intracranial pressure values and prevents brain tissue from being exposed to injury risks. The detection sensor further includes a first wireless power supply and communication module, which wirelessly powers internal components of the detection sensor and directly transmits measured parameters to an external receiver.

### FIELD

The present invention relates to the technical field of medical devices, in particular to an implantable pressure sensor and a monitoring sensor.

### BACKGROUND

Intracranial Pressure (ICP) is the pressure exerted by the intracranial contents on the cranial wall. Elevated intracranial pressure is a common manifestation of various intracranial diseases (such as traumatic brain injury, cerebral hemorrhage, brain tumors, etc.) or secondary intracranial injuries. Dynamic and continuous monitoring of intracranial pressure plays a crucial role in determining the patient's condition, guiding clinical medication, and evaluating prognosis.

Among existing invasive intracranial pressure monitoring technologies, a typical device uses a miniature MEMS pressure sensing chip to perform measurement by directly contacting brain tissue or cerebrospinal fluid. However, to achieve such direct-contact measurement and ensure its long-term, stable and safe operation in vivo, this technical approach faces severe challenges, which directly result in difficulties in reducing the size of the sensor's core components.

The main reason for its large size is that to meet biocompatibility and long-term implantation stability, and prevent signal drift, the sensing chip must be encapsulated in an extremely robust, hermetic, and bioinert housing (such as titanium package). The physical size and the structural complexity of this protective “package” are often much greater than those of the MEMS chip itself. The complex packaging structure is necessary to ensure the function and safety of the device, but inevitably results in a relatively large physical volume of the entire sensing unit during implantation, which increases the difficulty of surgical implantation, the patient's foreign body sensation, and the potential risk of compression to surrounding tissues.

Therefore, there is an urgent need in the art for an innovative pressure sensing scheme that can fundamentally eliminate the reliance on complex and robust packaging structures, thereby obtaining new solutions for achieving the miniaturization of implantable sensors, reducing implantation trauma, and improving patient comfort.

### SUMMARY

In view of the above problems, the present invention is proposed to provide an implantable pressure sensor and a monitoring sensor that overcome the above problems or at least partially address them.

In a first aspect, an embodiment of the present invention provides an implantable pressure sensor, comprising:
an inflatable bag for being implanted into a site to be measured;
a first gas pressure sensor disposed in an environment in gas communication with interior of the inflatable bag, for measuring an gas pressure value inside the inflatable bag.

In one embodiment, the inflatable bag has a first open end;
the implantable pressure sensor further comprises:
a first housing, inside which the first gas pressure sensor is located;
wherein the first housing has an open end connected to the first open end of the inflatable bag, so that interior of the first housing is in gas communication with the inflatable bag.

In one embodiment, the first housing has an open end connected to the first open end of the inflatable bag, so that interior of the first housing is in gas communication with the inflatable bag, which is further implemented as:
a conduit is disposed on a surface of the first housing, one open end of the conduit communicates with an space of the interior of the first housing, and the other open end communicates with the first open end of the inflatable bag.

In one embodiment, the first housing and the conduit are integrally formed.

In one embodiment, the sensor further comprises a support member, to which the first housing is fixed;
wherein the support member is further provided with a first microprocessor and a first wireless power supply and communication module;
the first wireless power supply and communication module is used to supply power to the first microprocessor and the first gas pressure sensor, and to communicate with an external receiver; and
the first microprocessor is used to control an operating state of the first gas pressure sensor, acquire the gas pressure value measured by the first gas pressure sensor, and perform a first preset processing on the gas pressure value measured by the first gas pressure sensor.

In one embodiment, the first preset processing comprises:
sending the gas pressure value measured by the first gas pressure sensor to an external receiver via the first wireless power supply and communication module; or,
determining a pressure value at the site to be measured according to the gas pressure value measured by the first gas pressure sensor, and sending the pressure value at the site to be measured to an external receiver via the first wireless power supply and communication module.

In one embodiment, the first wireless power supply and communication module comprises a first NFC chip and a receiving coil;
wherein the first NFC chip is used to receive energy emitted by an external receiver via the receiving coil and convert the energy into a preset voltage, so as to supply power to the first microprocessor and the first gas pressure sensor, and to communicate with a external device.

In one embodiment, the support member comprises an integrated circuit board on which the receiving coil is disposed.

In one embodiment, the sensor further comprises a support rod, one end of which passes through the first housing and is connected to the support member, and the other end of which extends into the inflatable bag.

In one embodiment, when the support member is an integrated circuit board:
the inflatable bag further has a second open end provided with a heat conductive plug;
the other end of the support rod extends through the inflatable bag and is connected to the heat conductive plug; and
a temperature sensor is disposed inside the heat conductive plug.

In one embodiment, the first microprocessor is further configured to acquire a temperature value monitored by the temperature sensor at the site to be measured, and send the temperature value at the site to be measured to an external receiver via the first wireless power supply and communication module.

In one embodiment, the implantable pressure sensor further comprises:
a second housing for covering and protecting the support member and components disposed on the support member.

In a second aspect, an embodiment of the present invention provides an implantable pressure monitoring sensor, comprising the implantable pressure sensor according to any one of the above and a receiver;
wherein the receiver comprises a processor and a second wireless power supply and communication module;
the second wireless power supply and communication module is used to cooperate with the first wireless power supply and communication module of the implantable pressure sensor to achieve supplying power to internal components of the implantable pressure sensor, and communicating with the first wireless power supply and communication module of the implantable pressure sensor to acquire parameter values measured by the implantable pressure sensor; and
the processor is used to perform a second preset processing on the parameter values.

In one embodiment, the parameter values comprise the gas pressure value measured by the first gas pressure sensor or the pressure value at the site to be measured; and
the second preset processing comprises:
when the parameter values comprise the gas pressure value measured by the first gas pressure sensor, determining the pressure value at the site to be measured according to the gas pressure value measured by the first gas pressure sensor.

In one embodiment, the second wireless power supply and communication module comprises a transmitting coil and a second NFC chip.

In one embodiment, the receiver further comprises a main body bearing part and a transmitting coil bearing part;
wherein the processor and the second NFC chip are disposed on the main body bearing part;
the transmitting coil is disposed on the transmitting coil bearing part;
the main body bearing part and the transmitting coil bearing part are separately arranged; and
components on the main body bearing part are electrically connected and/or signal-connected to the transmitting coil on the transmitting coil bearing part via a connecting member.

In one embodiment, the connecting member is electrically connected and/or signal-connected to the components on the main body bearing part in a detachable way;
and/or
the connecting member is electrically connected and/or signal-connected to the transmitting coil on the transmitting coil bearing part in a detachable way.

In one embodiment, the receiver further comprises a second gas pressure sensor for measuring atmospheric pressure; and
the when the parameter values comprise the gas pressure value measured by the first gas pressure sensor, determining the pressure value at the site to be measured
according to the gas pressure value measured by the first gas pressure sensor comprises:
when the parameter values comprise the gas pressure value measured by the first gas pressure sensor, determining the pressure value at the site to be measured according to the atmospheric pressure and the gas pressure value measured by the first gas pressure sensor.

In one embodiment, the receiver further comprises at least one of a wireless communication module and a display module; wherein
the wireless communication module is signal-connected to the processor and used to implement two-way communication between the receiver and an external terminal; and
the display module is used to perform a display operation under the control of the processor.

Compared with the prior art, the present invention has at least the following beneficial effects:
By adopting a corporation of an inflatable bag structure and a pressure sensor, volume and size of the detection sensor have been effectively reduced, while the risk of damage caused by direct contact between pressure sensors or other electronic components and tissues within the organism can be avoided by use of an inflatable bag structure. The detection sensor can further comprise a first wireless power supply and communication module, which realizes wireless power supply for internal components of the detection sensor and communication with external devices, resulting in a highly integrated structure of the detection sensor without a battery, further reducing the volume and size of the detection sensor and improving the user experience of the implant recipient, while ensuring the procession and communication performances of the detection sensor. Meanwhile, a drawback that wired detection sensors require continuous exposure of the wound during use is resolved, whereas after implantation of the detection sensor, the wound of the implant recipient can be sutured, which reduces the risk of clinical infection and restrictions on the postoperative activities of the implant recipient. The invention improves convenience operation of the detection sensor and expands application scenarios, and can provide real-time and reliable data for the diagnosis of diseases and injuries.

Other features and advantages of the present invention will be set forth in the following description, and in part will become apparent from the description, or may be learned by practice of the present invention. The objectives and other advantages of the present invention may be realized and attained by the structures particularly pointed out in the written description, claims, and accompanying drawings.

The technical solution of the present invention will be further described in detail below with reference to the accompanying drawings and embodiments.

### BRIEF DESCRIPTION OF THE DRAWINGS

The accompanying drawings are provided to facilitate a further understanding of the present invention, constitute a part of the description, and serve to explain the present invention together with the embodiments of the present invention, but shall not be construed as limiting the present invention. In the accompanying drawings:

FIG. 1 is a first structural schematic diagram of an implantable pressure sensor provided by an embodiment of the present invention.

FIG. 2 is a second structural schematic diagram of an implantable pressure sensor provided by an embodiment of the present invention.

FIG. 3 is a third structural schematic diagram of an implantable pressure sensor provided by an embodiment of the present invention.

FIG. 4 is a fourth structural schematic diagram of an implantable pressure sensor provided by an embodiment of the present invention.

FIG. 5 is a connection schematic diagram of internal components of an implantable pressure sensor provided by an embodiment of the present invention.

FIG. 6 is a principle schematic diagram of an implantable pressure sensor provided by an embodiment of the present invention.

FIG. 7 is an overall schematic diagram of an implantable pressure monitoring sensor provided by an embodiment of the present invention.

FIG. 8 is a data diagram of wireless energy transmission test results between a transmitting coil and a receiving coil in an implantable pressure monitoring sensor provided by an embodiment of the present invention.

FIG. 9 is a data diagram of test results of an implantable pressure monitoring sensor applied in a water tank experiment scenario provided by an embodiment of the present invention.

FIG. 10 is a data diagram of nonlinear error test results of an implantable pressure monitoring sensor provided by an embodiment of the present invention.

FIG. 11 is a structural schematic diagram of a support structure of an inflatable bag in an implantable pressure sensor provided by an embodiment of the present invention.

FIG. 12 is another structural schematic diagram of a support structure of an inflatable bag in an implantable pressure sensor provided by an embodiment of the present invention.

### REFERENCE NUMERALS

1. inflatable bag; 2. First gas pressure sensor; 3. First housing; 4. Plug; 5. Support rod; 6. Support member; 7. First microprocessor; 8. First wireless power supply and communication module; 9. Second housing; 10. Receiving coil; 11. First NFC chip.

### DETAILED DESCRIPTION OF EMBODIMENTS

Exemplary embodiments of the present disclosure will be described in more detail below with reference to the accompanying drawings. Although exemplary embodiments of the present disclosure are shown in the drawings, it should be understood that the present disclosure may be embodied in various forms and should not be limited to the embodiments set forth herein. Rather, these embodiments are provided for a thorough understanding of the present disclosure and to fully convey the scope of the present disclosure to those skilled in the art.

In the description of the present invention, it should be noted that the orientation or positional relationships indicated by the terms “center”, “upper”, “lower”, “left”, “right”, “vertical”, “horizontal”, “inner”, “outer”, “far”, “near”, “front”, “back”, etc., are based on the orientation or positional relationships shown in the accompanying drawings. They are merely for the convenience of describing the present invention and simplifying the description, rather than indicating or implying that the device or element referred to must have a specific orientation, be constructed and operated in a specific orientation, and therefore cannot be construed as limiting the present invention. In addition, the terms “first”, “second”, and “third” are used for descriptive purposes only and shall not be construed as indicating or implying relative importance.

In the description of the present invention, it should be noted that, unless otherwise explicitly specified and limited, the terms “mounted”, “connected”, and “coupled” shall be understood in a broad sense. For example, they may be fixed connections, detachable connections, or integral connections; they may be mechanical connections or electrical connections; they may be direct connections or indirect connections through an intermediate medium, and they may be internal communication between two elements. For those of ordinary skill in the art, the specific meanings of the above terms in the present invention can be understood according to the specific circumstances.

The terms “include”, “have” and any variations thereof in this application are intended to cover non-exclusive inclusions. For example, a process, method, system, product or device that comprises a series of steps or units is not limited to those listed steps or units, but may optionally include other steps or units not listed, or may optionally include other steps or units inherent to such process, method, product or device.

Reference to “an embodiment” herein means that a particular feature, structure or characteristic described in connection with the embodiment may be included in at least one embodiment of this application. The appearances of the phrase in various places in the specification are not necessarily all referring to the same embodiment, nor are separate or alternative embodiments mutually exclusive of other embodiments. It is understood explicitly and implicitly by those skilled in the art that the embodiments described herein can be combined with other embodiments.

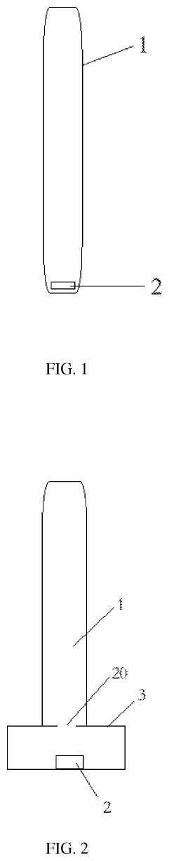

Refer to FIG. 1, it illustrates a first structure of an implantable pressure sensor provided by an embodiment of the present application. The implantable pressure sensor comprises an inflatable bag 1 and a pressure sensor. The inflatable bag 1 is used to be implanted into a site to be measured such that the inflatable bag 1 is in direct contact with the interior of the site to be measured, which may include, but is not limited to, the cerebral ventricle, bladder, etc. A first gas pressure sensor 2 is placed in an environment in gas communication with the interior of the inflatable bag 1, and is used to measure the gas pressure value inside the inflatable bag 1.

The inflatable bag 1 itself is flexible and deformable, and can be made of flexible materials such as rubber, so that when implanted into a site to be measured such as the cerebral ventricle or bladder, the inflatable bag 1 can deform under the liquid pressure of the liquid substance in the site to be measured, thereby changing the pressure of the gas environment inside the inflatable bag 1. The shape of the inflatable bag can be made as needed, for example, cylindrical, ellipsoidal, spherical, etc. An appropriate volume of gas can be pre-injected into the inflatable bag before the detection sensor leaves the factory or before use, and the volume of the injected gas can be set according to requirements, unnecessary to be inflated during surgery.

When the inflatable bag 1 is in direct contact with the site to be measured (e.g., in contact with cerebrospinal fluid in the cerebral ventricle or fluid in the bladder), fluid substance in the site to be measured exerts pressure on the outer wall of the inflatable bag 1 due to its own liquid pressure, causing the inflatable bag 1 to deform. At this time, the gas pressure inside the inflatable bag 1 is equal to the fluid pressure of the fluid substance in the site to be measured. Since the first gas pressure sensor 2 is in gas communication with the interior of the inflatable bag 1, the gas pressure value measured by the first gas pressure sensor 2 is the gas pressure value inside the inflatable bag 1, that is, the fluid pressure of the fluid substance in the site to be measured, which provides direct data support for the subsequent determination of the pressure value of the site to be measured.

By referring to the corresponding structure of the detection sensor formed by combining inflatable bag 1 and first gas pressure sensor 2, as shown in FIG. 1, it can be seen that no complex mechanical transmission or fluid guiding structure is required. This greatly reduces the number of components and the space occupied, while enabling the measurement of the fluid pressure of the fluid substances at the site to be measured with a minimal number of components. Consequently, it achieves the measurement of pressure values at the sites such as the cerebral ventricle and bladder, and effectively reduces the overall size of the detection sensor, achieving the miniaturization of the detection sensor while avoiding direct contact between the sensor or other electronic components and human tissues, thereby minimizing potential infection risks.

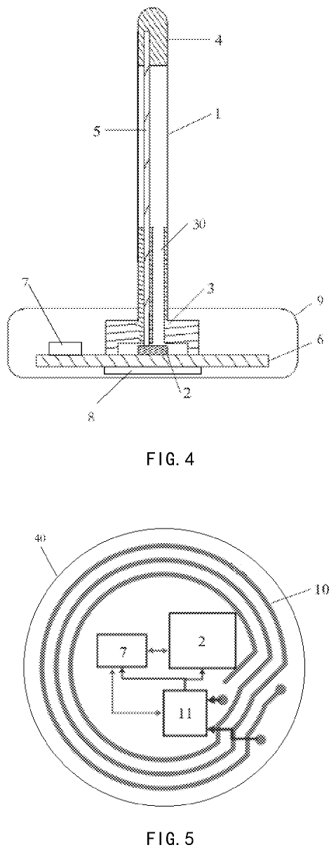

In one embodiment, for example, when the inflatable bag is a cylindrical inflatable bag tube as shown in FIG. 4, the diameter of the inflatable bag tube generally does not exceed 2.8 mm, and the length can be adjusted according to the age of the subject into whom it is to be implanted, usually ranging from 2 cm to 5 cm.

To enhance the detection performances of the pressure sensor, further improvements are made on the basis of the detection sensor of the first structure mentioned above, as detailed below.

In one embodiment, as shown in FIG. 2, the inflatable bag 1 has a first open end. In this case, the implantable pressure sensor further includes: a first housing 3, in which the first gas pressure sensor 2 is disposed; and an open end 20 provided on the first housing 3, and connected to the first open end of the inflatable bag 1, so that the interior of the first housing 3 is in gas communication with the inflatable bag 1, which can be connected as a sealed connection.

The first housing 3 completely encloses the first gas pressure sensor 3, and a certain space is reserved between the first housing 3 and the outer surface of the first gas pressure sensor 2 (so that the first gas pressure sensor 2 is in a gas environment communicating with the inflatable bag 1). Only one open end 20 is left on the first housing 3. The open end 20 is connected to the first open end of the inflatable bag 1, so that the interior of the first housing 3 is in gas communication with the inflatable bag 1. In this way, the gas pressure value measured by the first gas pressure sensor 2 is equal to the gas pressure value inside the inflatable bag 1. Preferably, the effective detection surface of the first gas pressure sensor 2 is preferably arranged toward the space formed between it and the first housing for gas accommodation, so that the effective detection surface of the first gas pressure sensor can fully contact the gas environment of the inflatable bag 1, improving the accuracy of gas pressure detection. The material of the first housing may be made of polycarbonate etc., for example.

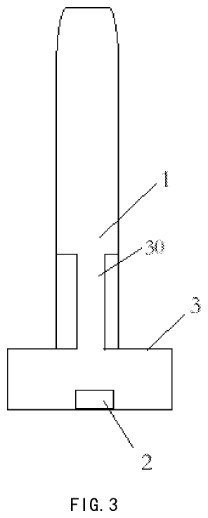

Specifically, as shown in FIG. 3, the above statement that “the first housing 3 has an open end 20, which is connected to the first open end of the inflatable bag 1, so that the interior of the first housing 3 is in gas communication with the inflatable bag 1” in FIG. 2 can be specifically implemented in the form shown in FIG. 3: a conduit 30 is arranged on the surface of the first housing 3. One open end of the conduit 30 is communicated with the inner space of the first housing 3, and the other open end is communicated with the first open end of the inflatable bag 1. Preferably, the first housing 3 and the conduit 30 are integrally formed, so as to ensure the airtightness of the connection between the first housing 3 and the conduit. The said conduit can be a hollow tubular structure, which serves as a communication medium to realize the communication between the inner space of the first housing 3 and the inner space of the inflatable bag 1.

The first open end of the inflatable bag 1 is hermetically connected to the other open end of the conduit, which can ensure the airtightness of the connection between the inflatable bag 1 and the conduit, avoid gas leakage at the connection between the inflatable bag 1 and the conduit, which will disturb the gas pressure in the internal space of the detection sensor and affect the detection accuracy of intracranial pressure.

In another embodiment, as shown in FIG. 4, the implantable pressure sensor may further comprise:
a support member 6, to which the first housing 3 is fixed, so as to improve the mechanical stability of the first housing 3;
a first microprocessor 7 and a first wireless power supply and communication module 8, provided on the support member 6;
wherein the first wireless power supply and communication module 8 is connected to the first microprocessor 7 and the first gas pressure sensor 2, and is capable of supplying power to the first microprocessor 7 and the first gas pressure sensor 2, and communicating with an external receiver 12. That is, the first wireless power supply and communication module 8 can realize the power supply for the internal components of the detection sensor and the communication function with external device in a wireless manner; and
wherein the first microprocessor 7 is used to control the operating states of the first gas pressure sensor 2, such as measurement frequency and switching actions, acquire the gas pressure value measured by the first gas pressure sensor 2, and perform a first preset processing on the gas pressure value measured by the first gas pressure sensor 2.

In one embodiment, the first preset processing may be implemented as follows: the gas pressure value measured by the first gas pressure sensor 2 is sent to the external receiver through the first wireless power supply and communication module 8; the external receiver determines the pressure value of the site to be measured according to the gas pressure value measured by the first gas pressure sensor 2.

Alternatively, the first preset processing may also be implemented as follows: determining the pressure value of the site to be measured according to the gas pressure value measured by the first gas pressure sensor 2; and then sending the pressure value of the site to be measured to the external receiver through the first wireless power supply and communication module 8. Here “determining the pressure value of the site to be measured according to the gas pressure value measured by the first gas pressure sensor 2” may be specifically implemented as follows: acquiring the atmospheric pressure of the current environment; calculating the difference between the gas pressure value measured by the first gas pressure sensor 2 and the atmospheric pressure, and the difference is the pressure value of the site to be measured, wherein the acquiring the atmospheric pressure of the current environment may be the detection sensor acquiring the atmospheric pressure of the current environment from other external devices via the first wireless power supply and communication module. The other devices here may be an external receiver or any other external devices capable of measuring atmospheric pressure, which is not limited herein.

In one embodiment, the first wireless power supply and communication module may comprise a first NFC chip and a receiving coil. The arrangement patterns and positions of these two components in the detection sensor can be adjusted as needed. The first NFC chip is configured to receive energy emitted by the external receiver via the receiving coil and convert it into a preset voltage, so as to supply power to the first microprocessor and the first gas pressure sensor, and communicate with external devices.

In a specific embodiment, as shown in FIG. 5, the support member 6 may be embodied as an integrated circuit board 40, and the integrated circuit board 40 is provided with a receiving coil 10; and a first microprocessor 7 in signal connection with a first gas pressure sensor 2 and a first NFC chip 11 respectively, wherein the receiving coil 10 is electrically connected to the first NFC chip 11, and transmits the received energy from the external receiver to the first NFC chip 11; and the first NFC chip 11 is electrically connected to the first microprocessor 7 and the first gas pressure sensor 2 respectively, so that the first NFC chip 11 converts the received energy into a preset voltage, thereby stably supplying power to the first microprocessor 7 and the first gas pressure sensor 2.

The structure corresponding to this embodiment avoids the provision of a battery in the detection sensor. Moreover, the wireless power supply and wireless communication modules can be reused, and the number of components of the entire detection sensor is small, thereby further reducing the volume of the detection sensor.

In another embodiment, as shown in FIG. 4, since the inflatable bag 1 is made of a flexible material, in order to provide the inflatable bag 1 with sufficient mechanical strength to be implanted into the site to be measured, the detection sensor may further comprise a support rod 5. One end of the support rod 5 passes through the first housing 3 and is connected to the support member 6, and the other end extends into the inflatable bag 1. The support rod 5 may be made of a material with sufficient hardness and toughness, such as titanium wire. The support rod 5 may have, but is not limited to, an elongated rod-like structure, which can provide sufficient support for the inflatable bag while reducing the spatial volume occupied by the support rod 5 inside the detection sensor to the greatest extent.

Furthermore, when the support member is embodied as the above-mentioned integrated circuit board, the inflatable bag 1 may further have a second open end, and the second open end is provided with a heat-conduction plug 4. The heat-conduction plug 4 seals the second open end. The first housing 3, the inflatable bag 1 and the heat-conduction plug 4 form a closed gas environment space. The heat-conduction plug may be made of metal or any other materials with a high thermal conductivity, which is not limited in the present application. At this time, the other end of the support rod extends through the inflatable bag and is connected to the heat-conduction plug, and a temperature sensor may be further disposed in the heat-conduction plug. When the detection sensor is implanted into the site to be measured, the heat-conduction plug 4 and the temperature sensor enable the measurement of the temperature value of the site to be measured.

Furthermore, the first microprocessor can acquire the temperature value of the site to be measured sensed by the temperature sensor, and transmit the temperature value to the first wireless power supply and communication module, so as to send the sensed temperature value of the site to be measured to the external receiver, realizing simultaneous measurement of the pressure value and temperature value of the site to be measured.

Preferably, in one embodiment, the support rod 5 may be embodied as a conductive support rod which is conductive (in this case, the support rod may be made of a conductive material, or the support rod can also be made of other materials with a conductive structure inside). One end of the conductive support rod is electrically connected to the circuit on the integrated circuit board, and the other end extends through the inflatable bag and is connected to the heat-conduction plug. The temperature sensor is electrically connected to the conductive support rod, so that the temperature sensor is connected to the first microprocessor on the integrated circuit board. This structure further highly integrates the functional devices of the detection sensor and reduces the volume dimensions of the detection sensor.

In addition, the implantable pressure sensor further comprises a second housing 9 for covering and protecting the support member 6 and the components disposed on the support member 6, so as to prevent the corresponding components from being damaged by collision during use of the detection sensor, and also prevent the components from directly contacting the implantable tissues, thereby avoiding the risk of infection.

The working principle of the implantable pressure sensor in the present application is described below.

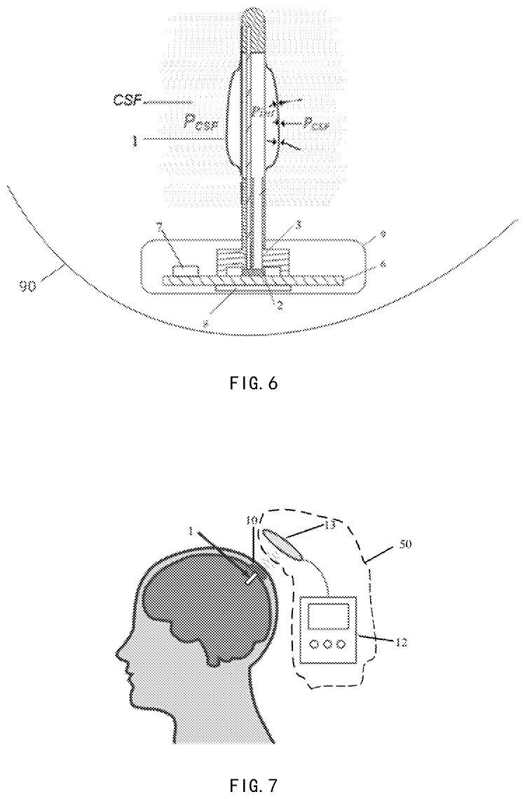

As shown in FIG. 6, the measurement principle of the implantable pressure sensor of the present application is as follows: the upper half of the detection sensor, i.e., the core control part including the support member, the first microprocessor, the first wireless power supply and communication module, etc., is placed flat in the gap between the outer surface of the skull and the scalp 90; and the components such as the inflatable bag, the conduct, and so on connected to the core control part are placed into the target monitoring region. After the inflatable bag 1 of the detection sensor is implanted into the target monitoring region, the outer wall of the inflatable bag 1 contacts the fluid substance in the target monitoring region, such as cerebrospinal fluid. At this time, the internal gas pressure P_{INT} of the inflatable bag 1 is equal to the absolute pressure P_{CSF} of the fluid substance in the target monitoring region. Since the first housing 3 and the inflatable bag 1 mutually form a gas communication environment, and the heat-conduction plug blocks the other end of the inflatable bag 1, a closed space inflated with gas is formed. Therefore, the absolute pressure Pₛₑₙₛ measured by the first gas pressure sensor 2 inside the first housing 3 is equal to the internal gas pressure P_{INT} inside the inflatable bag 1, i.e., the absolute pressure Pₛₑₙₛ measured by the first gas pressure sensor 2 is equal to the absolute pressure P_{CSF} of the target monitoring region.

In one embodiment, a support structure made of a three-dimensional porous material is further disposed in the inflatable bag 1, and the support structure has the following functions: preventing the inflatable bag membrane from non-linearly adhering to itself or wrinkling during pressure changes, so as to ensure good repeatability and linearity of the pressure-deformation relationship; accelerating pressure balance and improving response speed by maintaining a permanent, tiny internal space channel; ensuring that the opposite inner wall membranes of the inflatable bag do not adhere to itself so as to be blocked under negative pressure or low pressure, to prevent the inflatable bag from failing to function properly and avoid jump errors in the pressure value when the inflatable bag is restarted.

Specifically, the support structure may be implemented in the following two modes:

### Mode 1: Integral Liner

As shown in FIG. 11, a liner 01 is provided to be attached to the inner wall surface of the inflatable bag 1. The liner 01 is made of a porous material (such as polyurethane soft foam, polyvinyl alcohol hydrogel sponge, medical silica gel sponge, etc.), which is hollow and has a shape matching the internal shape of the inflatable bag. The thickness of the liner 01 is smaller than that of the inflatable bag 1. For example, the natural thickness of the inflatable bag is 1 mm, and the thickness of the support frame is 0.8 mm, which ensures that the liner is in a slightly compressed state inside the inflatable bag, and able to provide continuous support without over-expanding the inflatable bag and affecting its flexibility.

The manufacturing process of the liner may be implemented as follows:

Step 1: Place the liner on the core rod of the inflatable bag molding mold.

Step 2: Using the two-shot injection molding or dip molding process, the inflatable bag material (such as silica gel or polyurethane) in liquid is wrapped around the exterior of the liner, allowing the outer surface of the liner to be attached and integrated with the inner surface of the inflatable bag, thereby forming an inflatable bag with the above-mentioned liner arranged inside.

### Mode 2: Discrete Filling Particles

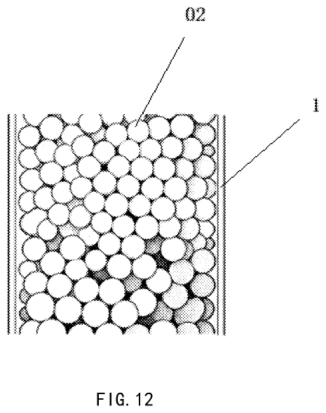

As shown in FIG. 12, discrete filling particles 02 are made from a three-dimensional porous material. The number of the discrete filling particles is multiple, and their shapes are spheres, cylinders, etc. The size of the discrete filling particles should be determined according to the cavity size of the inflatable bag, which should be not only small enough to ensure that they can be filled into the inflatable bag to form a particle group and achieve a sufficient number, but also not too small to cause particle agglomeration due to van der Waals force.

For example, the diameter of the particles may be in the range of [a*D,b*D], where D is the width (or diameter) of the cavity of the inflatable bag, a is 1/50, and b is 1/20.

The discrete filling particles 02 as a support structure are filled in the inflatable bag 1, and the filling number can be determined as needed.

The discrete filling particles form an omnidirectional dead-angle-free dynamic support network through a large number of randomly distributed tiny particles, fundamentally eliminating the problem of the inner membrane of the inflatable bag adhered to itself, and significantly improving the linearity and repeatability of measurement. Moreover, their isotropic mechanical response ensures the stability and accuracy of pressure measurements. In addition, in engineering implementation, this solution is insensitive to manufacturing errors and able to be adapted to various complex inflatable bag cavity shapes, which greatly reduces the difficulty and the cost of the manufacturing process, and the natural gaps between the discrete filling particles form an efficient three-dimensional flow channel, which ensures a rapid pressure balance and response speed.

As shown in FIG. 7, the present application further provides an implantable pressure monitoring sensor, comprising the above-mentioned implantable pressure sensor and a receiver 50. The receiver 50 comprises a processor and a second wireless power supply and communication module. The second wireless power supply and communication module is used to cooperate with the first wireless power supply and communication module of the implantable pressure sensor to able to supply power to the internal components of the implantable pressure sensor and communicate with the first wireless power supply and communication module of the implantable pressure sensor to obtain the parameter values measured by the implantable pressure sensor. The processor is used to perform a second preset processing on the parameter values.

In one embodiment, the second wireless power supply and communication module can comprise a transmitting coil and a second NFC chip. Correspondingly, the first wireless power supply and communication module can be embodied as a receiving coil and a first NFC chip. Certainly, a power supply module such as a battery is provided in the receiver for supplying power to itself. The processor controls the power supply module to provide energy to the transmitting coil. Energy is transmitted between the transmitting coil and the receiving coil through electromagnetic induction, and then wireless power supply to the components in the detection sensor is realized by using the first NFC chip.

In one embodiment, the parameter r values measured by the above-mentioned implantable pressure sensor and obtained by the receiver can include the gas pressure value measured by the first gas pressure sensor or the pressure value of the site to be measured.

When the parameter values include the gas pressure value measured by the first gas pressure sensor, the above-mentioned second preset processing can be specifically implemented as follows: acquiring the atmospheric pressure of the current environment; calculating the difference between the gas pressure value measured by the first gas pressure sensor and the atmospheric pressure of the current environment, with the value of the difference giving the pressure value of the site to be measured.

Preferably, the above-mentioned acquiring the atmospheric pressure of the current environment can be implemented as follows: acquiring the atmospheric pressure of the current environment from other external devices through the second wireless power supply and communication module. Alternatively, the above-mentioned receiver itself measures the atmospheric pressure of the current environment. In this case, the receiver can further comprise a second gas pressure sensor for measuring the atmospheric pressure.

In one embodiment, to facilitate wireless power supply to the detection sensor implanted in a organism, as shown in FIG. 7, the receiver 50 further comprises a main body bearing part 12 (such as a housing) and a transmitting coil bearing part 13 (such as a circuit board integrated with a transmitting coil). A processor and a second NFC chip are disposed on the main body bearing part, a transmitting coil is disposed on the transmitting coil bearing part, and the main body bearing part and the transmitting coil bearing part are separately disposed. The components on the main body bearing part are electrically connected and/or signal-connected with the transmitting coil on the transmitting coil bearing part through a connecting part. The connecting part can be detachably and electrically connected and/or signal-connected with the components on the main body bearing part, or detachably and electrically connected and/or signal-connected with the transmitting coil on the transmitting coil bearing part, thereby facilitating use and storage. The connecting part may be a detachable connecting part, such as a transmission line with pluggable connectors or interfaces at both ends. Correspondingly, the connector (or interface) at one end of the transmission line connected to the components on the main body bearing part of the receiver is adapted to the interface (or connector) disposed on the main body bearing part; and the connector (or interface) at the other end of the transmission line connected to the transmitting coil on the transmitting coil bearing part is adapted to the interface (or connector) disposed on the transmitting coil bearing part. In addition, the connecting part can also be fixedly connected to the transmitting coil on the transmitting coil bearing part, and the end connected to the components on the main body bearing part of the receiver is provided with a pluggable interface (or connector). Correspondingly, a corresponding adapted pluggable connector (or interface) is provided on the main body bearing part of the receiver. The length of the transmission line can be set as needed.

The method of use of the above-mentioned receiver and detection sensor is illustrated as follows. As shown in FIG. 7, the upper half of the implantable pressure sensor (i.e., the support member provided with the first microprocessor, and the first wireless power supply and communication module etc.) is placed in a organism (such as between the lateral side of the skull and the subcutaneous tissue), and the lower half (inflatable bag part) is implanted in a organism (such as inserted into the brain parenchyma or ventricle of the organism through a skull opening. An inflatable bag of an appropriate length is selected for matching as needed). After implantation, the scalp on the skull is sutured. The transmitting coil bearing part of the receiver is fixed on the outer side of the scalp layer through a bandage or other means, so that the transmitting coil of the receiver and the receiving coil of the detection sensor are in a concentric position, and the total transcutaneous distance does not exceed a specified distance, so as to ensure the energy transmission efficiency between the transmitting coil and the receiving coil.

In one embodiment, the receiver further comprises at least one of a wireless communication module and a display module, wherein the wireless communication module is in signal connection with the processor and is used to realize two-way communication between the receiver and an external terminal; and the display module is used to perform a display operation under the control of the processor, thereby realizing visual display of the pressure value of the site to be measured.

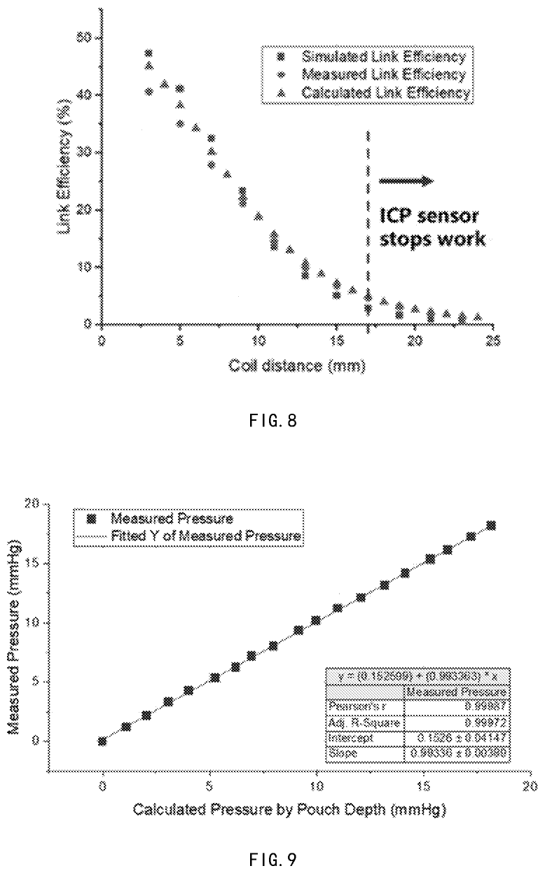

As shown in FIG. 8, it is a data diagram of the wireless energy transmission test results between the transmitting coil and the receiving coil in the implantable pressure monitoring sensor of the present invention. In the wireless energy transmission test, the receiving coil was implanted between 3 mm pig skin and 5 mm pig tenderloin to simulate the human implantation environment, and the wireless power transmission efficiency (PTE) between the transmitting coil and the receiving coil was tested. It can be seen from FIG. 8 that the maximum working distance between the transmitting coil and the receiving coil reaches 17 mm, that is, within a distance range of less than or equal to 17 mm, stable and efficient wireless energy transmission can be achieved between the transmitting coil and the receiving coil.

As shown in FIG. 9, it is a data diagram of the test results of the implantable pressure monitoring sensor of the present invention used in a water tank experiment scenario. In the water tank experiment scenario, a water tank experimental device is built to provide a controllable in-vitro experimental environment for the implantable pressure monitoring sensor, so as to verify the feasibility of the implantable pressure monitoring sensor for fluid pressure measurement. Specifically, the implantable pressure sensor of the implantable pressure monitoring sensor is placed in a water tank, the underwater depth of the implantable pressure sensor inside the water tank is adjusted, and the underwater depth where the implantable pressure sensor is located is measured using a ruler or other tools. According to the measured underwater depth, the water pressure intensity at the position of the implantable pressure sensor is calculated, and the water pressure measured by the implantable pressure monitoring sensor itself is obtained. The test results in FIG. 9 shows that within the range of 0~18 mmHg, the differences between the measured water pressure and the calculated water pressure are less than ±0.1 mmHg.

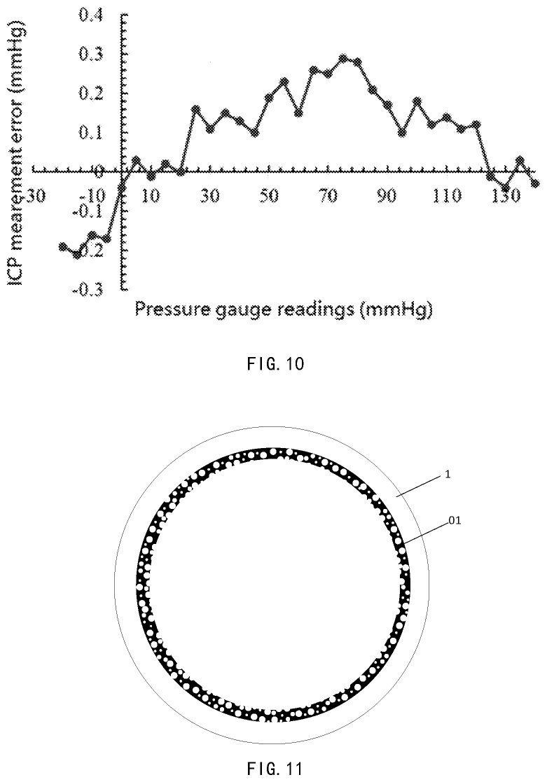

As shown in FIG. 10, it is a data diagram of the full-scale nonlinear error test results of the implantable pressure monitoring sensor of the present invention. It can be seen from FIG. 10 that the test range is −20~150 mmHg, and the measurement nonlinear errors are less than ±0.3 mmHg within the entire test range.

The above-mentioned implantable pressure monitoring sensor provided by the embodiments of the present invention cooperates with the miniaturized and highly integrated detection sensor to jointly realize safe, accurate and convenient monitoring of the pressure in a organism. The system uses wireless energy transmission technology to stably supply power to the implantable sensor and receive the pressure and temperature data measured by it. It not only inherits and demonstrates the core advantages of the sensor such as small size, battery-free and elimination of tissue damage, but also converts the original signal of the sensor into directly usable clinical information through the intelligent processing and display functions at the receiver end. Finally, the system realizes a complete closed loop from implantable sensing to in-vitro display, while ensuring high precision and high stability, which greatly improves the comfort for the implant recipient and the safety and convenience for the clinical operations.

To summarize, the above-mentioned implantable pressure sensor and monitoring sensor provided by the embodiments of the present invention can obtain the fluid pressure value at the site to be measured by combining the inflatable bag with the first gas pressure sensor, which provides a data basis for the subsequent determination of the pressure value at the site to be measured. By adopting a combination of an inflatable bag structure and a pressure sensor, the volume of the detection sensor has been effectively reduced. Meanwhile, the use of the inflatable bag structure can avoid the risk of damage caused by direct contact between pressure sensors or other electronic components and tissues within the organism. The above-mentioned detection sensor can further comprise the first wireless power supply and communication module, so as to enable wireless power supply to the internal components of the detection sensor and communication with the external devices, resulting in the structure of the detection sensor highly integrated without the need for a battery, which further reduces the size of the detection sensor, improves the user experience of the implant recipient, ensures the processing performance and communication performance of the detection sensor, improves the operational convenience of the detection sensor, and provides a real-time and reliable data basis for judging diseases and injuries.

Although the preferred embodiments of the present invention have been described, those skilled in the art can make additional changes and modifications to these embodiments once they learn of the basic inventive concepts. Therefore, the appended claims are intended to be construed to include the preferred embodiments and all changes and modifications that fall within the scope of the present invention.

Obviously, those skilled in the art can make various changes and modifications to the embodiments of the present invention without departing from the spirit and scope of the embodiments of the present invention. Thus, if these modifications and variations of the embodiments of the present invention fall within the scope of the claims of the present invention and their equivalent technologies, the present invention also intends to include these modifications and variations.

What is claimed is:

1. An implantable pressure sensor, comprising:
an inflatable bag for being implanted into a site to be measured; and
a first gas pressure sensor disposed in an environment in gas communication with interior of the inflatable bag, for measuring an gas pressure value inside the inflatable bag.

2. The implantable pressure sensor according to claim 1, wherein
the inflatable bag has a first open end; and
the implantable pressure sensor further comprises:
a first housing, in which the first gas pressure sensor is disposed; and
where in the first housing has an open end connected to the first open end of the inflatable bag, so that interior of the first housing is in gas communication with the inflatable bag.

3. The implantable pressure sensor according to claim 2, wherein
the first housing has an open end connected to the first open end of the inflatable bag, so that the interior of the first housing is in gas communication with the inflatable bag, is further implemented as:
a conduit is disposed on a surface of the first housing, one open end of the conduit communicates with an space of interior of the first housing, and the other open end communicates with the first open end of the inflatable bag.

4. The implantable pressure sensor according to claim 3, wherein
the first housing and the conduit are integrally formed.

5. The implantable pressure sensor according to claim 2, further comprising:
a support member, to which the first housing is fixed; and
a first microprocessor and a first wireless power supply and communication module, provided on the support member;
wherein the first wireless power supply and communication module is used to supply power to the first microprocessor and the first gas pressure sensor, and to communicate with an external receiver; and
wherein the first microprocessor is used to control an operating state of the first gas pressure sensor, acquire the gas pressure value measured by the first gas pressure sensor, and perform a first preset processing on the gas pressure value measured by the first gas pressure sensor.

6. The implantable pressure sensor according to claim 5, wherein
the first preset processing comprises:
sending the gas pressure value measured by the first gas pressure sensor to an external receiver via the first wireless power supply and communication module; or,
determining a pressure value at the site to be measured according to the gas pressure value measured by the first gas pressure sensor, and sending the pressure value at the site to be measured to an external receiver via the first wireless power supply and communication module.

7. The implantable pressure sensor according to claim 5, wherein
the first wireless power supply and communication module comprises a first NFC chip and a receiving coil; and
wherein the first NFC chip is used to receive energy emitted by an external receiver via the receiving coil and convert the energy into a preset voltage, so as to supply power to the first microprocessor and the first gas pressure sensor, and to communicate with a external device.

8. The implantable pressure sensor according to claim 7, wherein
the support member comprises an integrated circuit board, on which the receiving coil is disposed.

9. The implantable pressure sensor according to claim 5, further comprising
a support rod, one end of which passes through the first housing and is connected to the support member, and the other end of which extends into the inflatable bag.

10. The implantable pressure sensor according to claim 9, wherein
when the support member is an integrated circuit board:
the inflatable bag further has a second open end provided with a heat conductive plug;
the other end of the support rod extends through the inflatable bag and is connected to the heat conductive plug; and
a temperature sensor is disposed inside the heat conductive plug.

11. The implantable pressure sensor according to claim 10, wherein
the first microprocessor is further used to acquire a temperature value monitored by the temperature sensor at the site to be measured, and send the temperature value at the site to be measured to an external receiver via the first wireless power supply and communication module.

12. The implantable pressure sensor according to claim 5, further comprising:
a second housing for covering and protecting the support member and components disposed on the support member.

13. The implantable pressure sensor according to claim 1, wherein
a support structure made of a three-dimensional porous material is further disposed in the inflatable bag;
the support structure is embodied as: a liner attached to an inner wall surface of the inflatable bag; and
the liner is made of a three-dimensional porous material, which is hollow, has a shape matching an inner shape of the inflatable bag, and has a thickness smaller than that of the inflatable bag.

14. An implantable pressure monitoring sensor, comprising the implantable pressure sensor according to claim 5, and a receiver, wherein
the receiver comprises a processor and a second wireless power supply and communication module;
the second wireless power supply and communication module is used to cooperate with the first wireless power supply and communication module of the implantable pressure sensor to achieve supplying power to internal components of the implantable pressure sensor, and communicating with the first wireless power supply and communication module of the implantable pressure sensor to acquire parameter values measured by the implantable pressure sensor; and
the processor is used to perform a second preset processing on the parameter values.

15. The implantable pressure monitoring sensor according to claim 14, wherein
the parameter values comprise the gas pressure value measured by the first gas pressure sensor or the pressure value at the site to be measured; and
the second preset processing comprises:
when the parameter values comprise the gas pressure value measured by the first gas pressure sensor, determining the pressure value at the site to be measured according to the gas pressure value measured by the first gas pressure sensor.

16. The implantable pressure monitoring sensor according to claim 14, wherein
the second wireless power supply and communication module comprises a transmitting coil and a second NFC chip.

17. The implantable pressure monitoring sensor according to claim 16, wherein
the receiver further comprises a main body bearing part and a transmitting coil bearing part; and
wherein the processor and the second NFC chip are disposed on the main body bearing part;
the transmitting coil is disposed on the transmitting coil bearing part;
the main body bearing part and the transmitting coil bearing part are separately arranged; and
components on the main body bearing part are electrically connected and/or signal-connected to the transmitting coil on the transmitting coil bearing part via a connecting member.

18. The implantable pressure monitoring sensor according to claim 17, wherein
the connecting member is electrically connected and/or signal-connected to the components on the main body bearing part in a detachable way; and/or
the connecting member is electrically connected and/or signal-connected to the transmitting coil on the transmitting coil bearing part in a detachable way.

19. The implantable pressure monitoring sensor according to claim 15, wherein
the receiver further comprises a second gas pressure sensor for measuring atmospheric pressure; and
the when the parameter values comprise the gas pressure value measured by the first gas pressure sensor, determining the pressure value at the site to be measured according to the gas pressure value measured by the first gas pressure sensor comprises:
when the parameter values comprise the gas pressure value measured by the first gas pressure sensor, determining the pressure value at the site to be measured according to the atmospheric pressure and the gas pressure value measured by the first gas pressure sensor.

20. The implantable pressure monitoring sensor according to claim 14, wherein
the receiver further comprises at least one of a wireless communication module and a display module; and
wherein
the wireless communication module is signal-connected to the processor and used to achieve two-way communication between the receiver and an external terminal; and
the display module is used to perform a display operation under the control of the processor.
